(19) United States
(12) Patent Application Publication
(10) Pub. No.: US 2024/0324638 A1
(43) Pub. Date: Oct. 3, 2024

(54) Solutions for impotence.

(71) Applicant: Menni Menashe Zinger, Ashkeloh (IL)

(72) Inventor: Menni Menashe Zinger, Ashkeloh (IL)

(21) Appl. No.: 18/129,903

(22) Filed: Apr. 3, 2023

(51) Int. Cl. A23L 19/00; A23G 1/50; A61K 31/496; A61K 33/30

(52) U.S. Cl. CPC A23L 19/09; A23G 1/50; A61K 31/496; A61K 33/30

(57) ABSTRACT

At this invention I suggest solutions to impotence.

At 2003, eating lemon pulp caused erecting after few hours at night at the best level to someone with problems and difficulties at having erecting because of using medications for mental problems and also a past surgeries at pines that damaged the nerves at pines.

At 2007 eating chocolate chips caused great erecting short time after words and very successful sex with a woman to same person above.

At 2011, using the medication abilify caused after 3 days of use at law to normal dosage, to very good erecting to same person above.

At 2009, using zinc caused a poisoned person that the poisoning shrank his pines and caused him shames to be again with normal size at pines at time with no erecting and improved erecting and at this dosage of 50-100 milligrams a day it is safe for mounts and after stop taking the zinc for one mount or two it is possible to take the zinc again for few mounts and so on.

1. using lemon pulp ingredients in order to improve erecting/erection at people with erecting difficulties and at the general population who wants better sex life by taking a tablet/pill/capsule/powder/cream/gel/drops 30 minutes-2 hours before sex.

2. using chocolate chips ingredients in order to improve erecting/erection at people with erecting difficulties and at the general population who wants better sex life by taking a tablet/pill/capsule/powder/cream/gel/drops 20 minutes-2 hours before sex.

3. using abilify medication at a dosage that is used for mental patients or at the law grades for that usage ingredients in order to improve erecting/erection at people with erecting difficulties and at the general population who wants better sex life by taking a tablet/pill/capsule/powder/cream/gel/drops for 3 days before sex and after the 3 days the ability to have erecting erection should be exist without other medications or help if continuing talking the abilify at normal dosage as written above here and this medication can be taken on regular base for mounts and even years.

4. using zinc supplement as medication in order to improve erecting/erection and in order to improve size of pines at normal stage and erecting too at dosage of 50-100 milligrams a day and at that dosage it is safe for mounts.
